(19) United States
(12) Patent Application Publication
(10) Pub. No.: US 2008/0140055 A1
(43) Pub. Date: Jun. 12, 2008

(54) LOCKABLE ENTERAL FEEDING ADAPTER

(75) Inventor: Ben D. Shirley, Salt Lake City, UT (US)

Correspondence Address: KIRTON AND MCCONKIE, 60 EAST SOUTH TEMPLE,, SUITE 1800, SALT LAKE CITY, UT, 84111, US

(73) Assignee: UTAH MEDICAL PRODUCTS, INC., Salt Lake City, UT (US)

(21) Appl. No.: 11/608,573

(22) Filed: Dec. 8, 2006

(51) Int. Cl. A61M 25/18

(52) U.S. Cl. 604/535; 604/538

(57) ABSTRACT

Lockable enteral feeding adapters are disclosed. A female adapter has a female tapered cavity having a circular sidewall, a tapered inner surface and an external surface. The female tapered cavity has an external opening at a proximal end thereof that does not form a liquid-tight connection with a standard male Luer conical fitting. One or more threads are disposed on the external surface of the tapered cavity. A male adapter has a male tapered cone is sized to mate with the female tapered cavity. The male tapered cone is too large to form an effective connection with a standard female Luer conical fitting. An internally threaded locking ring is mounted around the male tapered cone. Rotation of the locking ring relative to the one or more threads locks the male adapter to the corresponding female adapter to prevent unintentional disengagement of the male and female adapters.

### BACKGROUND OF THE INVENTION

This invention relates to a lockable adapter or fitting for use with enteral feeding systems and oral dose syringes. The invention further relates to an adaptor for use with enteral feeding systems and oral dose syringes that prevents coupling to an intravenous (“IV”) infusion line or standard Luer conical fitting.

Tubing and catheter misconnection errors are an important and potentially deadly problem in heath care facilities. One type of tube and catheter misconnection error involves enteral feeding tubes and intravenous catheters. Enteral feeding tubes, such as nasogastric feeding tubes, are used to administer liquid nutritional solutions and medications directly to a patient's gastrointestinal system. In contrast, intravenous catheters are used to administer liquid nutritional solutions and medications directly to a patient's vascular system. Serious injury and even death can occur when substances designed for enteral administration are administered intravenously. Patients may also be harmed when IV solutions are administered enterally, although the potential for serious injury or death is not as great as when enteral solutions are administered intravenously.

For example, there are regular reports of infants who accidentally received breast milk intravenously instead of through a nasogastric feeding tube. Errors such as this occur because of medical professionals using similar or identical tubing for different or unintended purposes. Luer connectors are implicated in or contribute to many of these errors because they enable functionally dissimilar tubes or catheters to be connected.

Luer connectors are widely used in connectors for medical tubing, catheters, and syringes. Luer is an abbreviation for what is commonly referred to as a Luer Taper, named for the 19th century German medical instrument maker, Hermann Wulfing Luer. Luer Taper is a standard system of tapers used for making leak-proof connections between a male conical fitting and its mating female part on medical and laboratory instruments, including syringes, needles, catheters, tubing, adapters, and so forth. There are two types of Luer conical fittings. The simpler one, a slip fitting, consists of a tapered cone and a mating tapered cavity. The Luer slip fitting holds the parts together by friction. A more secure fitting is achieved by adding locking threads to the two halves of a Luer slip fitting, forming a Luer lock fitting; this is the more popular of the two. Where two medical devices that carry small volumes of liquids must be joined, the Luer conical fitting is the most common means of achieving a leak-free junction.

One reason for their widespread use is because the size and shape of Luer connectors is standardized. The International Organization for Standards (ISO) has established standard ISO 594 which specifies the requirements for standard 6% Luer conical fittings for use with hyperdemic syringes and needles and with certain other apparatus for medical use such as transfusion and infusion sets.

An ISO standard female Luer conical fitting has a 6% tapered cavity with an external opening at a proximal end thereof. According to ISO standard 594, the external opening has a maximum inner diameter of 0.170 inches (4.315 mm) and a minimum inner diameter of 0.168 inches (4.270 mm); this corresponds to a standard external opening size of 0.169±0.001 inches. The external opening has an interior tapered diameter, measured at a distance of 0.300 inches distal from the external opening, in the range from 0.150 inches (3.812 mm) to 0.152 inches (3.858 mm).

An ISO standard male Luer conical fitting has a 6% tapered cone having a distal end thereof. The male conical fitting has a maximum external diameter at the distal end of 0.157 inches (3.990 mm) and a minimum external diameter of 0.1545 inches (3.925 mm); this corresponds to a standard external diameter at the distal end of about 0.156±0.001 inches. The male conical fitting has an exterior flared diameter, measured at a distance of 0.300 inches proximal from the distal end, in the range from 0.1725 inches (4.382 mm) to 0.177 inches (4.484 mm).

In contrast, there is presently no ISO standard size and shape connector for enteral feeding systems and oral dose syringe tips. However, enteral feeding systems and oral dose syringes are often intentionally designed to use adaptors and fittings that are larger than standard Luer fittings. Despite size differences, there are incidents of tubing misconnection errors where enteral and IV systems are similar in design and are equipped with similar components, making it possible for even trained medical professionals to confuse one for the other. In addition, because infusion sets and mating enteral feeding adapters are made in varying sizes, it is common to have disengagement between the adapter body and the enteral feeding tube. Because of the loose engagement, the feeding tube can be easily pulled from the adapter. This can inhibit proper patient treatment.

Thus, it would be an improvement in the art to provide a lockable adapter or fitting for use with enteral feeding systems and oral dose syringes to prevent unintentional disengagement. It would be a further improvement in the art to provide an adaptor for use with enteral feeding systems and oral dose syringes that prevents coupling to an IV infusion line or standard Luer conical fitting. Such an adapter is provided herein.

### BRIEF SUMMARY OF THE INVENTION

The present invention provides a lockable enteral feeding adapter having male and female parts. The female adapter has a female tapered cavity having a circular sidewall, a tapered inner surface and an external surface. The female tapered cavity has an external opening at a proximal end thereof and a tapered inner surface. The external opening has an inner diameter that is too large to form a liquid-tight connection with a standard male Luer conical fitting. Means for locking the female adapter to a corresponding male adapter may be disposed on the external surface of the tapered cavity. The locking means may include one or more threads. A variety of thread configurations are possible, including full threads, threads disposed on a radially extending flange, two short lugs, and variations of the foregoing.

The male adapter is sized and configured to mate with the female adapter. The male adapter has a male tapered cone having a circular sidewall, a tapered outer surface and an inner bore. The male tapered cone has a distal end with an external diameter and an outer surface that flares outwardly in a proximal direction from the distal end. The male tapered cone is too large to form a liquid-tight connection with a standard female Luer conical fitting. An internally threaded locking ring may be mounted around the male tapered cone, such that rotation of the locking ring relative to the locking means locks the male adapter to the corresponding female adapter to prevent unintentional disengagement of the male and female adapters. The locking ring may optionally be fabricated such that it has freedom to rotate about the male tapered cone. It may also be fabricated such that it has limited freedom to translate along the longitudinal axis of the male adapter.

The female tapered cavity and the male tapered cone are preferably fabricated with a 6% taper. Even though a 6% taper is the same taper used in standard Luer conical fittings, the female tapered cavity and male tapered cone have a sufficiently larger size to be incompatible with standard Luer conical fittings. In addition, the female adapter preferably includes at least one protrusion disposed at a distal end of the tapered inner surface to physically prevent complete insertion and coupling of a standard male Luer conical fitting.

The lockable enteral feeding adapters within the scope of the present invention are preferably connected to or configured to be connected to flexible tubing, such as enteral feeding catheters and feeding tubes. The adapters may be configured for direct bonding or attachment of flexible tubing. The adapters may be configured with structures, such as a barb connector, to facilitate connection to flexible tubing. The components of the lockable enteral feeding adapters may be fabricated of a variety of materials suitable for medical and health care applications.

These features and advantages of the present invention will become more fully apparent from the following description and appended claims, or may be learned by the practice of the invention as set forth hereinafter.

### BRIEF DESCRIPTION OF THE SEVERAL VIEWS OF THE DRAWINGS

In order that the manner in which the above-recited and other features and advantages of the invention are obtained will be readily understood, a more particular description of the invention briefly described above will be rendered by reference to specific embodiments thereof that are illustrated in the appended drawings. Understanding that these drawings depict only typical embodiments of the invention and are not therefore to be considered to be limiting of its scope, the invention will be described and explained with additional specificity and detail through the use of the accompanying drawings in which:

FIG. 1A is a perspective view of a female adapter within the scope of the present invention.

FIG. 1B is a side view of the female adapter shown in FIG. 1A.

FIG. 1C is an end view of the female adapter shown in FIG. 1A.

FIG. 1D is a cross-sectional view of the female adapter taken along line A-A of FIG. 1B.

FIG. 2A is an end view of a male adapter.

FIG. 2B is a cross-sectional view of the male adapter taken along line B-B of FIG. 2B.

FIG. 3 is a perspective view of a female adapter having locking threads in the form of lugs.

FIG. 4 is side view of a female adapter connected to an enteral feeding catheter.

FIG. 5 is a side view of enteral feeding extension tubing having female and male adapters connected thereto.

### DETAILED DESCRIPTION OF THE INVENTION

Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

Furthermore, the described features, structures, or characteristics of the invention may be combined in any suitable manner in one or more embodiments. In the following description, numerous specific details are provided, such as examples of cells, membranes, processes, methods, etc., to provide a thorough understanding of embodiments of the invention. One skilled in the relevant art will recognize, however, that the invention may be practiced without one or more of the specific details or method steps, or with other methods, components, materials, and so forth. In other instances, well-known structures, materials, or operations are not shown or described in detail to avoid obscuring aspects of the invention.

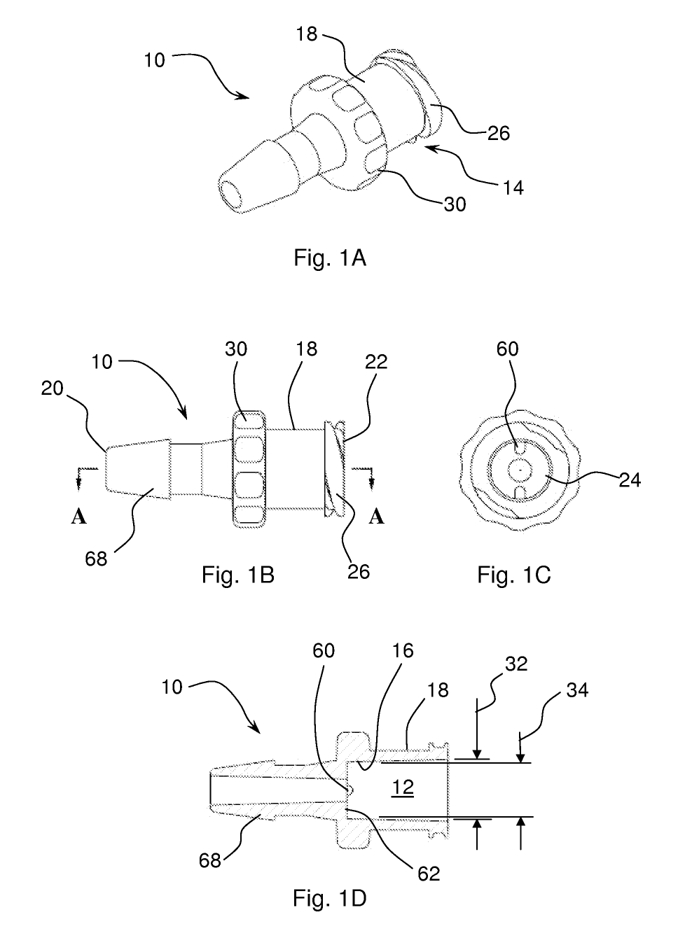

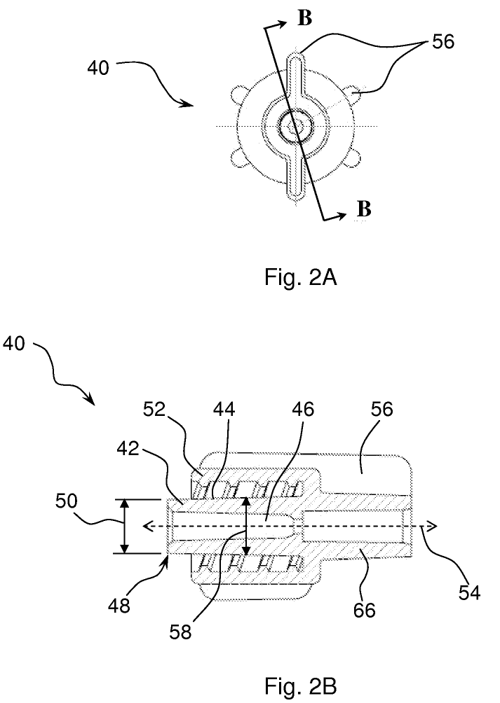

The embodiments of the present invention will be best understood by reference to the drawings, wherein like parts are designated by like numerals throughout. It will be readily understood that the components of the present invention, as generally described and illustrated in the figures herein, could be arranged and designed in a wide variety of different configurations. Thus, the following more detailed description of the embodiments of the lockable enteral feeding adapter as represented in FIGS. 1A-2B, is not intended to limit the scope of the invention, as claimed, but is merely representative of the embodiments of the invention.

The present invention is drawn to a lockable enteral feeding adapter having male and female parts. One possible female adapter 10 is shown in FIGS. 1A-1D. As shown in FIG. 1D, the female adapter 10 has a female tapered cavity 12 having a circular sidewall 14, a tapered inner surface 16, and an external surface 18. The female adapter 10 has a distal end 20 and a proximal end 22. The female tapered cavity 12 has an external opening 24 at the proximal end 22.

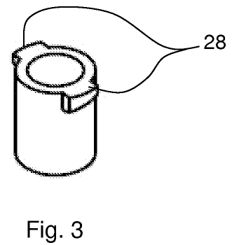

Means for locking the female adapter to a corresponding male adapter may be disposed on the external surface 18 of the tapered cavity 12. As shown in FIGS. 1A-1D, the locking means 26 may include one or more threads. A variety of thread configurations are possible, including full threads, threads disposed on a radially extending flange, as in FIGS. 1A-1D, two short lugs, and variations of the foregoing. FIG. 3 shows a locking means thread configuration in the form of two short lugs 28. Gripping surfaces 30 may be provided to facilitate handling and rotating the female adapter 10.

The external opening 24 has an inner diameter 32 that is too large to form a liquid-tight connection with a standard male Luer conical fitting. The external opening 24 has an inner diameter 32 in the range from 0.171 and 0.255 inches, and more preferably in the range from 0.185 and 0.215 inches. In one embodiment, the external opening 24 of the female tapered cavity 12 has an inner diameter 32 of 0.196±0.001 inches. The female tapered cavity 12 has an interior tapered diameter 34, measured at a distance of 0.300 inches distal from the external opening 24, in the range from 0.157 to 0.241 inches, and more preferably in the range from 0.177 to 0.197 inches. In one embodiment, the female tapered cavity 12 has an interior tapered diameter 34, measured at a distance of 0.300 inches distal from the external opening 22, of 0.180±0.001 inches.

One possible male adapter 40 is shown in FIGS. 2A-2B. The male adapter 40 is sized and configured to mate with the female adapter 10. The male adapter 40 has a male tapered cone 42 having a circular sidewall, a tapered outer surface 44 and an inner bore 46. The male tapered cone 42 has a distal end 48 with an external diameter 50. The outer surface 44 flares outwardly in a proximal direction from the distal end 48.

An internally threaded locking ring 52 may be mounted around the male tapered cone 42, such that rotation of the locking ring 52 relative to the locking means locks the male adapter 40 to the corresponding female adapter 10 to prevent unintentional disengagement of the male and female adapters. The locking ring 52 may optionally be fabricated such that it has freedom to rotate about the male tapered cone. It may also be fabricated such that it has limited freedom to translate along the longitudinal axis 54 of the male adapter 40. Gripping surfaces 56 may be provided to facilitate handling and rotating of the male adapter 40

The male tapered cone 42 is too large to form a liquid-tight connection with a standard female Luer conical fitting. The distal end 48 of the male tapered cone 42 has an external diameter 50 in the range from 0.158 to 0.241 inches. In one embodiment, the distal end 48 of the male tapered cone 42 has an external diameter 50 from about 0.175 to 0.195 inches. In one embodiment, the distal end 48 of the male tapered cone 42 has an external diameter 50 of 0.185±0.001 inches. The male tapered cone 52 has a flared diameter 56, measured at a distance of 0.300 inches proximal from the distal end 48, in the range from 0.172 to 0.255 inches. In one embodiment, the flared diameter 58, measured at a distance of 0.300 inches, ranges from about 0.190 to 0.210 inches. In one embodiment, the male tapered cone 52 has a flared diameter 56, measured at a distance of 0.300 inches proximal from the distal end 48, of 0.199±0.001 inches.

The female tapered cavity 12 and the male tapered cone 42 are sized and configured to permit slip fit connection with most commercially available oral dose enteral feeding catheters, tubes, and syringes. In this manner, the female adapter and male adapter may be used with non-locking adapters in a slip fit connection. However, to forming a locking connection, the female and male adapters should have complementary locking means, such as those described herein.

The female tapered cavity 12 and the male tapered cone 42 are preferably fabricated with a 6% taper. The 6% taper provides effective fluid-tight coupling between the female and male adapters. Even though a 6% taper is the same taper used in standard Luer conical fittings, the female tapered cavity 12 and male tapered cone 42 are incompatible with standard Luer conical fittings. This is due to size incompatibility. In addition, the female adapter 10 preferably includes at least one protrusion 60 disposed at a distal end 62 of the female tapered cavity 12 to physically prevent complete insertion and coupling of a standard male Luer conical fitting.

The lockable enteral feeding adapters within the scope of the present invention are preferably connected to or configured to be connected to flexible tubing, such as enteral feeding catheters and feeding tubes. The adapters may be configured for direct bonding or attachment of flexible tubing. For example, the male adapter 40 shown in FIG. 2B includes a tubing receiver 66 sized and configured to receive flexible tubing. The flexible tubing may be bonded to the tubing receiver 66. The adapters may be configured with structures, such as a barb connector 68, shown in FIG. 1B, to facilitate connection to flexible tubing.

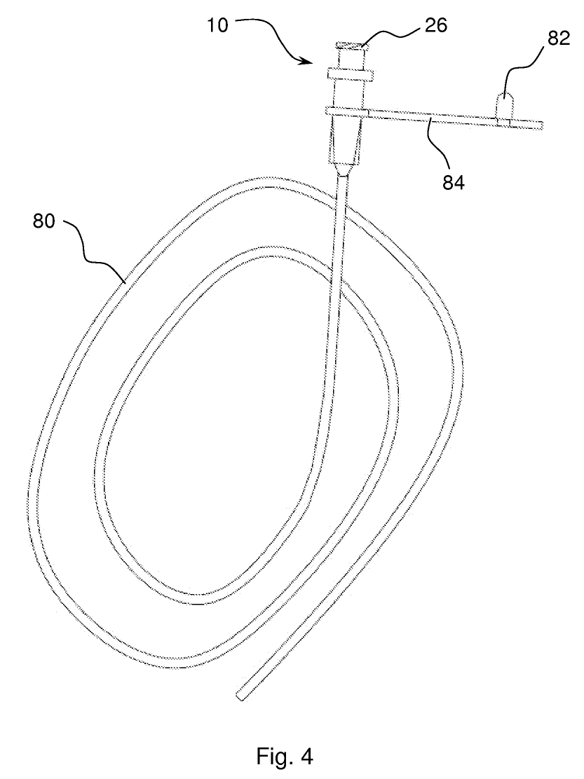

In one embodiment shown in FIG. 4, a female adapter 10 is connected to an enteral feeding catheter 80, such as a nasogastric NICU catheter. One example of a useful enteral feeding catheter 80 is the NUTRI-CATH® catheter manufactured by Utah Medical Products, Inc., Midvale, Utah. The catheter is manufactured of medical grade soft silicone and may be used for enteral feeds, gastric sampling, and/or oral medication administration. A cap 82 sized to fit within the female tapered cavity of the female adapter 10 may be connected to a flexible tab 84. If desired, the cap 82 may close and seal the female adapter 10.

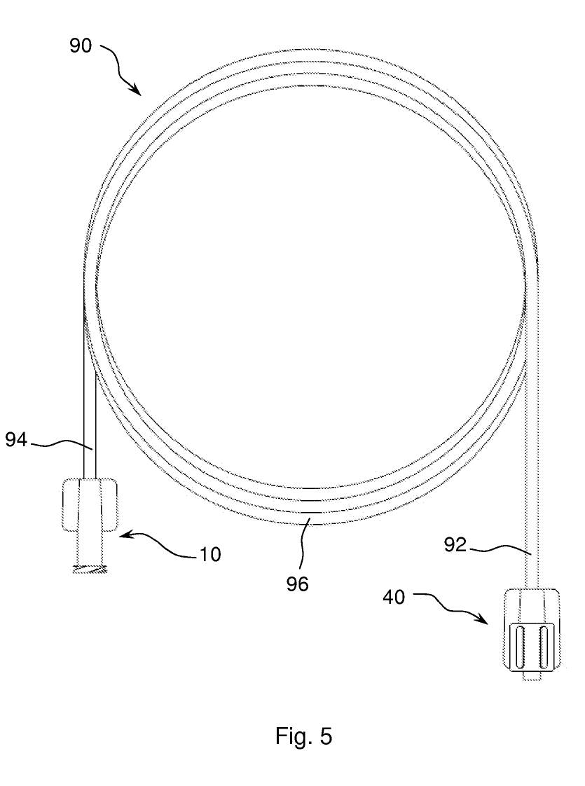

In another embodiment shown in FIG. 5, a male adapter 40 and a female adapter 10 may be connected to an enteral feeding extension set 90. The male adapter 40 may be disposed at distal end 92 and the female adapter may be disposed at a proximal end 94. The enteral feeding extension set 90 comprises medical grade flexible tubing 96. While the tubing 96 is shown coiled in FIG. 5, it will be appreciated that the flexible tubing may be uncoiled and extended the full length of the tubing 96. In use, the male adapter 40 may be connected to a female adapter 10 associated with an enteral feeding catheter, such as catheter 80 shown in FIG. 4. The female adapter 10 may be connected to a source of enteral fluid for nutrition or medication according to the needs of the patient.

The components of the lockable enteral feeding adapters may be fabricated of a variety of materials suitable for medical and health care applications. For example, the female or male adapters may be fabricated from a medical grade material such as, but not limited to, nylon, polypropylene (PP), polycarbonate (PC), polyvinylidene fluoride (PVDF), acrylonitrile butadiene styrene (ABS), and polyvinyl chloride (PVC).

The ability to lock the male and female adapters ensures that the connection will not accidentally slip apart. As mentioned above, the male and female adapters are sized and configured to prevent attachment to standard Luer conical fittings. In this way, inadvertent enteral feeding through standard IV tubing is avoided.

While specific embodiments of the present invention have been illustrated and described, numerous modifications come to mind without significantly departing from the spirit of the invention, and the scope of protection is only limited by the scope of the accompanying claims.

1. Lockable enteral feeding adapters comprising:
a female adapter comprising a female tapered cavity having a circular sidewall, a tapered inner surface and an external surface, said female tapered cavity having an external opening at a proximal end thereof, said external opening having an inner diameter and tapered inner surface that is too large to form a liquid-tight connection with a standard male Luer conical fitting;
one or more threads disposed on the external surface of the tapered cavity;
a male adapter sized to mate with the female adapter comprising a male tapered cone having a circular sidewall, a tapered outer surface and an inner bore, said male tapered cone having a distal end with an external diameter and tapered outer surface that is too large to form a liquid-tight connection with a standard female Luer conical fitting; and
an internally threaded locking ring mounted around the male tapered cone, wherein rotation of the locking ring relative to the one or more threads locks the male adapter to the corresponding female adapter to prevent unintentional disengagement of the male and female adapters.

2. The lockable enteral feeding adapters according to claim 1, further comprising at least one protrusion disposed at a distal end of the tapered inner surface to prevent complete insertion and coupling of a standard male Luer conical fitting.

3. The lockable enteral feeding adapters according to claim 1, wherein the female tapered cavity is connected to or configured to be connected to flexible tubing.

4. The lockable enteral feeding adapters according to claim 3, wherein the flexible tubing comprises an enteral feeding catheter.

5. The lockable enteral feeding adapters according to claim 1, wherein the male tapered cone is connected to or configured to be connected to flexible tubing.

6. The lockable enteral feeding adapters according to claim 1, wherein the external opening of the female tapered cavity has an inner diameter in the range from 0.171 and 0.255 inches and an interior tapered diameter, measured at a distance of 0.300 inches distal from the external opening, in the range from 0.157 to 0.241 inches.

7. The lockable enteral feeding adapters according to claim 1, wherein the external opening of the female tapered cavity has an inner diameter in the range from 0.195 and 0.215 inches and an interior tapered diameter, measured at a distance of 0.300 inches distal from the external opening, in the range from 0.177 to 0.197 inches.

8. The lockable enteral feeding adapters according to claim 1, wherein the distal end of the male tapered cone has an external diameter in the range from 0.158 to 0.241 inches and a flared diameter, measured at a distance of 0.300 inches proximal from the distal end, in the range from 0.172 to 0.255 inches.

9. The lockable enteral feeding adapters according to claim 1, wherein the distal end of the male tapered cone has an external diameter in the range from 0.185±0.010 inches and a flared diameter, measured at a distance of 0.300 inches proximal from the distal end, in the range from 0.199±0.010 inches.

10. The lockable enteral feeding adapters according to claim 1, wherein the female adapter or male adapter is fabricated from a material selected from nylon, polypropylene (PP), polycarbonate (PC), polyvinylidene fluoride (PVDF), acrylonitrile butadiene styrene (ABS), and polyvinyl chloride (PVC).

11. A female adapter for use with an enteral feeding system comprising:
a female tapered cavity having a circular sidewall, a tapered inner surface and an external surface, said female tapered cavity having an external opening at a proximal end thereof, said external opening having an inner diameter and tapered inner surface that is too large to form a liquid-tight connection with a standard male Luer conical fitting; and
means for locking the female adapter to a corresponding male adapter, said means for locking being disposed on the external surface.

12. The female adapter according to claim 11, wherein the external opening of the female tapered cavity has an inner diameter in the range from 0.171 and 0.255 inches and an interior tapered diameter, measured at a distance of 0.300 inches distal from the external opening, in the range from 0.157 to 0.241 inches.

13. The female adapter according to claim 11, wherein the external opening of the female tapered cavity has an inner diameter in the range from 0.185 and 0.215 inches and an interior tapered diameter, measured at a distance of 0.300 inches distal from the external opening, in the range from 0.177 to 0.197 inches.

14. The female adapter according to claim 11, further comprising at least one protrusion disposed at a distal end of the tapered inner surface to prevent complete insertion and coupling of a standard male Luer conical fitting.

15. The female adapter according to claim 11, wherein the female tapered cavity is connected to or configured to be connected to flexible tubing.

16. The female adapter according to claim 15, wherein the flexible tubing comprises an enteral feeding catheter.

17. The female adapter according to claim 11, wherein the means for locking comprises one or more threads.

18. The female adapter according to claim 17, wherein the one or more threads are disposed on a radially extending flange.

19. The female adapter according to claim 11, wherein the female tapered cavity is fabricated from a material selected from nylon, polypropylene (PP), polycarbonate (PC), polyvinylidene fluoride (PVDF), acrylonitrile butadiene styrene (ABS), and polyvinyl chloride (PVC).

20. The female adapter according to claim 11, further comprising a barb connector disposed at a distal end of the female tapered cavity for connection to flexible tubing.

21. A male adapter for use with an enteral feeding system comprising:
a male tapered cone having a circular sidewall, a tapered outer surface and an inner bore, said male tapered cone having a distal end with an external diameter and tapered outer surface that is too large to form a liquid-tight connection with a standard female Luer conical fitting; and
means for locking the male adapter to a corresponding female adapter.

22. The male adapter for use with an enteral feeding system according to claim 21, wherein the distal end of the male tapered cone has an external diameter in the range from 0.158 to 0.241 inches and a flared diameter, measured at a distance of 0.300 inches proximal from the distal end, in the range from 0.172 to 0.255 inches.

23. The male adapter for use with an enteral feeding system according to claim 21, wherein the distal end of the male tapered cone has an external diameter in the range from 0.185±0.010 inches and a flared diameter, measured at a distance of 0.300 inches proximal from the distal end, in the range from 0.199±0.010 inches.

24. The male adapter for use with an enteral feeding system according to claim 21, wherein the male tapered cone is connected to or configured to be connected to flexible tubing.

25. The male adapter for use with an enteral feeding system according to claim 24, wherein the flexible tubing comprises an enteral feeding catheter.

26. The male adapter for use with an enteral feeding system according to claim 21, wherein the male tapered cone is fabricated from a material selected from nylon, polypropylene (PP), polycarbonate (PC), polyvinylidene fluoride (PVDF), acrylonitrile butadiene styrene (ABS), and polyvinyl chloride (PVC).

27. The male adapter for use with an enteral feeding system according to claim 21, further comprising a barb connector disposed at a proximal end of the male tapered cone for connection to flexible tubing.

28. The male adapter for use with an enteral feeding system according to claim 21, wherein the means for locking comprises an internally threaded locking ring mounted around the male tapered cone.

29. The male adapter for use with an enteral feeding system according to claim 28, whereby rotation of the locking ring relative to the female adapter locks the male adapter to the corresponding female adapter.

30. The male adapter for use with an enteral feeding system according to claim 28, wherein the male adapter has a longitudinal axis and wherein the locking ring has freedom to rotate about the male tapered cone and limited freedom to translate along the longitudinal axis.

31. A female adapter for use with an enteral feeding system comprising:
a female tapered cavity having a circular sidewall, a tapered inner surface and an external surface, said female tapered cavity having an external opening at a proximal end thereof, said external opening having an inner diameter and tapered inner surface that is too large to form a liquid-tight connection with a standard male Luer conical fitting; and
at least one protrusion disposed at a distal end of the tapered inner surface to prevent complete insertion and coupling of a standard male Luer conical fitting.

32. The female adapter according to claim 31, wherein the external opening of the female tapered cavity has an inner diameter in the range from 0.171 and 0.255 inches and an interior tapered diameter, measured at a distance of 0.300 inches distal from the external opening, in the range from 0.157 to 0.241 inches.

33. The female adapter according to claim 31, wherein the external opening of the female tapered cavity has an inner diameter in the range from 0.195 and 0.215 inches and an interior tapered diameter, measured at a distance of 0.300 inches distal from the external opening, in the range from 0.177 to 0.197 inches.
